(19) United States
(12) Patent Application Publication
(10) Pub. No.: US 2025/0387540 A1
(43) Pub. Date: Dec. 25, 2025

(54) COMPOSITION FOR INDUCING LIGAMENTIZATION OF TENDON, COMPRISING ANTERIOR CRUCIATE LIGAMENT-DERIVED STEM CELLS

(71) Applicant: IUCF-HYU INDUSTRY-UNIVERSITY COOPERATION FOUNDATION HANYANG UNIVERITY, Seoul (KR)

(72) Inventors: Jin Kyu LEE, Seoul (KR); Jin Sung PARK, Seoul (KR); Sung Sin JO, Uijeongbu-si (KR)

(73) Assignee: IUCF-HYU INDUSTRY-UNIVERSITY COOPERATION FOUNDATION HANYANG UNIVERITY, Seoul (KR)

(21) Appl. No.: 18/880,365

(22) PCT Filed: Jun. 30, 2023
(86) PCT No.: PCT/KR2023/009233

§ 371 Date: Dec. 31, 2024

(30) Foreign Application Priority Data
Jul. 1, 2022 (KR) 10-2022-0081326
May 12, 2023 (KR) 10-2023-0061661

(51) Int. Cl. A61L 27/38; A61L 27/24; A61L 27/36; A61L 27/54

(52) U.S. Cl. CPC A61L 27/3834; A61L 27/24; A61L 27/3662; A61L 27/54; A61L 2300/252; A61L 2300/64; A61L 2430/10

(57) ABSTRACT

The present invention relates to a composition for inducing ligamentization of tendons, comprising anterior cruciate ligament-derived stem cells. The use of the composition can inhibit fibrosis in tendon grafts used for anterior cruciate ligament reconstruction surgeries and promote the healing process. Additionally, the composition may further include extracellular matrix proteins of the cruciate ligament tissue.

### TECHNICAL FIELD

The present invention relates to a composition for inducing ligamentization of tendons, including anterior cruciate ligament-derived stem cells, and the composition may further include extracellular matrix proteins of cruciate ligament tissue.

### BACKGROUND ART

At the center of the knee joint, there are the anterior cruciate ligament (ACL) in the front and the posterior cruciate ligament (PCL) in the back in the form of a cross. The anterior cruciate ligament plays a role in supporting the tibia (shin bone, lower bone of the knee joint), which forms the knee joint, such that it does not slip forward relative to the femur (thigh bone, upper bone of the knee joint). The anterior cruciate ligament is easily damaged by strong external forces, and an anterior cruciate ligament rupture occurs while the knee is twisted or bent in the motion of landing after jumping during exercise, when running fast and suddenly stopping, or when suddenly changing directions.

The treatment method after an anterior cruciate ligament rupture depends on the degree of knee displacement, instability, age, and activity level. Since people with partial rupture of the anterior cruciate ligament, people who are relatively old (over 60 years old), office workers with low activity levels, people who rarely engage in sports, and the like often do not feel any movement in their knee joints in their daily lives, they can live while improving knee function and preventing further injury through rehabilitation such as muscular strengthening exercises.

However, when the movement (instability) of the knee is severe, it may cause inconvenience in daily life and damage to the articular cartilage or meniscus may lead to the rapid progression of arthritis, so surgery is required. In particular, when a patient has a complete rupture and is young and actively engaged in active work or sports, surgical treatment is required because additional damage to other ligaments, articular cartilage, meniscus cartilage, and the like may occur even with aggressive rehabilitation treatment, causing a sense of instability and inconvenience in daily life.

An anterior cruciate ligament reconstruction surgery is usually performed as a surgical treatment for anterior cruciate ligament rupture, and is a surgical procedure in which tunnels are created in the femur and tibia, and then passing a tendon graft through the tunnels and fixing the tendon graft. However, the anterior cruciate ligament reconstruction surgery has limitations, such as the tendon healing process taking 12 to 24 months or more in the human body and degeneration into fibrous tissue (scar tissue, type III collagen) that is weaker than normal anterior cruciate ligament tissue due to the tendon fibrosis that occurs during the healing process. Further, due to the degeneration, the tendon graft exhibits less than 50% of the biomechanical strength of normal ligaments. Therefore, young men have a high rate of ligament re-rupture even after an anterior cruciate ligament reconstruction surgery.

Webster et al. reported that in patients aged 20 years or younger who underwent an anterior cruciate ligament reconstruction surgery, the re-rupture rate reached about 33.5% at an average follow-up of 3.4 years (Scale. Orthop J Sports Med 2021 August 18; 9(8)), and Beischer et al. reported that patient groups who returned to sports before 9 months after an anterior cruciate ligament reconstruction surgery had a re-rupture rate of the tendon graft that was 7-fold or more than those who returned to sports later (J Orthop Sports Phys Ther. 2020 February: 50 (2): 83-90).

### DISCLOSURE

### Technical Problem

Under the circumstances described above, the present inventors studied a method capable of inhibiting fibrosis in tendon grafts used for anterior cruciate ligament reconstruction surgeries to shorten the healing process and increase the mechanical strength of the tendon grafts, and have devised a method for injecting anterior cruciate ligament tissue-derived stem cells and a cruciate ligament extracellular matrix-derived collagen complex together into the tendon grafts (FIG. 1). When the above method is used, stem cells have excellent engraftment and survival, are well integrated into a tendon matrix, and have excellent type I collagen production. These results mean that tendon grafts may be subjected to a normal healing process of the ligament instead of fibrosis.

Therefore, an object of the present invention is to provide a composition for inducing ligamentization of tendons, including anterior cruciate ligament-derived stem cells as an active ingredient.

In addition, another object of the present invention is to provide a pharmaceutical composition for treating anterior cruciate ligament rupture, including a tendon for a graft; and anterior cruciate ligament-derived stem cells injected into the tendon for a graft as active ingredients.

### Technical Solution

In order to achieve the above objects, an aspect of the present invention provides a composition for inducing ligamentization of tendons, including anterior cruciate ligament-derived stem cells as an active ingredient.

As used herein, ‘tendon’ refers to a connective tissue that connects muscles and bones and transmits the force generated by muscles to bones to cause joint movement, and Type I collagen accounts for 85 to 95% of the weight of tendons, type III collagen accounts for about 5% or less thereof, and proteoglycan accounts for about 5% or less thereof. Furthermore, fibronectin, elastin, and the like provide a firm cell scaffold in tissue.

As used herein, ‘ligament’ refers to a fibrous connective tissue located at a connection site between bones, and plays a role in maintaining the stability of joints. At the center of the knee joint, there are the anterior cruciate ligament (ACL) in the front and the posterior cruciate ligament (PCL) in the back in the form of a cross, and the anterior cruciate ligament is easily ruptured by strong external forces.

As used herein, ‘composition for inducing ligamentization of a tendon for a graft’ refers to a composition that inhibits fibrosis in tendon grafts, produces type I collagen, and promotes the tendon healing process of the tendons to promote or induce the ligamentization of the tendons, when the tendon grafts used for anterior cruciate ligament reconstruction surgeries are implanted in the body.

As already described, even though a tendon is implanted into the knee joint, the anterior cruciate ligament reconstruction surgery has problems such as it takes a long time for the healing process of the tendon graft and the tendon graft turns into fibrous tissue that has weaker biomechanical strength than normal anterior cruciate ligament tissue due to fibrosis in the tendon generated during the healing process.

To solve these problems, studies have been conducted to confirm the effectiveness of adipose-derived mesenchymal stem cells (ADMSCs) and bone marrow-derived stem cells (BMSCs), which may be easily collected, when simply injecting them into the joint cavity or injecting them alone (without mixing with collagen) into the tendon graft during an anterior cruciate ligament reconstruction surgery, but the results were skeptical.

Thus, the present inventors devised a method for using stem cells derived from autologous or allogeneic anterior cruciate ligaments. An autologous anterior cruciate ligament refers to an anterior cruciate ligament that is isolated from a patient with an anterior cruciate ligament rupture, and an allogeneic anterior cruciate ligament refers to an anterior cruciate ligament that is isolated from someone other than the patient.

According to an exemplary embodiment of the present invention, ‘anterior cruciate ligament-derived stem cells’ may be obtained by the following process:
i) collecting a portion of anterior cruciate ligament tissue from an individual;
ii) dissolving an extracellular matrix in the collected anterior cruciate ligament tissue;
iii) passing an anterior cruciate ligament tissue solution in which the extracellular matrix is dissolved through a cell strainer; and
iv) recovering only cells which have passed through the cell strainer.

According to an exemplary embodiment of the present invention, in Step i) above, the individual may be a patient with a ruptured anterior cruciate ligament, and the collected anterior cruciate ligament tissue may be a portion of the ruptured tissue. The dissolving of the extracellular matrix may be carried out by immersing the collected anterior cruciate ligament tissue in a solution including type I collagenase. Step iii) is a step of removing unnecessary materials other than stem cells, and the cell strainer may have a mesh size of 70 μm. Only the cells that have passed through the cell strainer may be collected to obtain anterior cruciate ligament-derived stem cells. The obtained anterior cruciate ligament-derived stem cells are stored at −80° C. until the experiment, and may be proliferated and used, if necessary.

Among stem cells originating from the anterior cruciate ligament, CD34-positive stem cells are not stem cells derived from the tissue itself, but are hematopoietic origin stem cells derived from the surrounding blood vessels, and only a very small amount of cells can be collected from actual patients with ruptured anterior cruciate ligaments, and these cells are known to be completely different cells from mesenchymal stem cells (MSCs).

According to an exemplary embodiment of the present invention, the anterior cruciate ligament-derived stem cells used in the present invention are CD34-negative.

Meanwhile, there have been existing studies using commercialized type I collagen derived from animal tails or skin during anterior cruciate ligament reconstruction surgery, but the effect has been insignificant. Thus, the present inventors mixed a collagen complex extracted directly from the extracellular matrix of cruciate ligaments (anterior and posterior cruciate ligaments) tissue with stem cells derived from the anterior cruciate ligament, injected the resulting mixture into tendon grafts, and then confirmed the effect. As a result, it was confirmed that an experimental group in which a mixture of anterior cruciate ligament tissue-derived stem cells and a collagen complex was injected had the best engraftment and survival of stem cells and type I collagen production compared to experimental groups into which only anterior cruciate ligament-derived stem cells, only type I collagen, or only the collagen complex was injected (FIGS. 6, 8, and 9).

Therefore, the composition for inducing ligamentization of a tendon for a graft may include a collagen complex derived from allogeneic cruciate ligament tissue as an additional component. The collagen complex includes type I collagen as a main component.

In the present invention, the composition for inducing ligamentization of a tendon for a graft may further include components for culturing anterior cruciate ligament-derived stem cells and maintaining the function of the stem cells. As an example, the composition may further include dimethyl sulfoxide (DMSO), which is a commonly used cell cryoprotectant, and may include a basal medium commonly used in cell culture. The basal medium may be selected from the group consisting of Dulbecco's modified Eagle's medium (DMEM), 80% knockout DMEM, minimal essential medium (MEM), basal medium Eagle (BME), RPMI 1640, F-10, F-12, DMEM-F12, α-minimal essential medium (α-MEM), Glasgow's minimal essential medium (G-MEM), Iscove's modified Dulbecco's medium (IMDM), MacCoy's 5A medium, AmnioMax Medium, and Chang's Medium Mesem Cult-XF Medium, and can be used without limitation as long as it is any medium for culturing stem cells used in the art.

Furthermore, the composition for inducing ligamentization of a tendon for a graft of the present invention may further include components useful for cell culture, as long as it does not affect the function of anterior cruciate ligament-derived stem cells.

Another aspect of the present invention provides a composition for assisting ligamentization of a tendon for a graft, including a collagen complex derived from allogeneic cruciate ligament tissue.

In the present invention, the composition for assisting ligamentization of a tendon for a graft refers to a composition that is unlikely to induce ligamentization of the tendon for a graft alone, but is capable of ultimately promoting ligamentization of tendons by increasing the function of inducing/promoting ligamentization of stem cells or other substances.

In the composition for assisting ligamentization of a tendon for a graft, the ‘collagen complex’ is as described above.

The composition for assisting ligamentization of a tendon for a graft of the present invention may further include components such as a buffer solution and appropriate salts to prevent denaturation and degradation of the collagen complex.

Meanwhile, the inventors of the present invention confirmed that the engraftment, survival and type I collagen production of stem cells injected into the tendon for a graft were better than those of adipose-derived mesenchymal stem cells (ADMSCs) injected into the tendon for a graft (FIGS. 3, 4, 16, and 17). Since type I collagen is the main component of ligaments, its production in large amounts is advantageous for the ligamentization of tendons. Therefore, still another object of the present invention provides a pharmaceutical composition for treating anterior cruciate ligament rupture, including a tendon for a graft; and anterior cruciate ligament-derived stem cells injected into the tendon for a graft as active ingredients.

In addition, when anterior cruciate ligament-derived stem cells+an anterior cruciate ligament-derived extracellular matrix collagen complex were injected into an anterior cruciate ligament rupture animal model, the degree of tendon ligamentization and collagen I formation was better than that of the control (FIGS. 13 to 15).

Therefore, the pharmaceutical composition may further include a collagen complex derived from the allogeneic cruciate ligament, and the collagen complex includes collagen I as the main component.

According to an exemplary embodiment of the present invention, the anterior cruciate ligament-derived stem cells may be injected into the transitional zone of the tendon for a graft through a syringe in the form of a cell suspension, and when a collagen complex is further included, a mixture of the anterior cruciate ligament-derived stem cells and the collagen complex may be injected into the tendon for a graft through a syringe.

In the pharmaceutical composition for treating anterior cruciate ligament rupture, the ‘anterior cruciate ligament-derived stem cells’ and the ‘cruciate ligament-derived collagen complex’ are as described above. The tendon for a graft is autologous or allogeneic, autologous tendons are those taken from a person himself/herself, and allogeneic tendons refer to tendons obtained from a donated cadaver.

The pharmaceutical composition of the present invention may further include components for culturing anterior cruciate ligament-derived stem cells and maintaining the function of the stem cells, as described above for the composition for inducing ligamentization of a tendon for a graft, and may further include a pharmaceutically acceptable carrier. A pharmaceutically acceptable carrier included in the composition is a pharmaceutically acceptable carrier typically used in the manufacture of preparations, and includes lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, and the like, but is not limited thereto.

Meanwhile, yet another aspect of the present invention provides a method for treating anterior cruciate ligament rupture or regenerating an anterior cruciate ligament, the method including administering the above-described pharmaceutical composition to a subject in need thereof.

The subject refers to all animals, including humans, monkeys, cows, horses, sheep, pigs, cats and dogs, in which an anterior cruciate ligament rupture has occurred or may occur.

### Advantageous Effects

When the composition for inducing ligamentization of the present invention is used, it is possible to inhibit fibrosis in tendon grafts used for anterior cruciate ligament reconstruction surgeries and induce and promote a normal ligament healing process.

### DESCRIPTION OF DRAWINGS

FIG. 1 is a schematic view of the implantation of a mixture of autologous anterior cruciate ligament-derived stem cells and cruciate ligament-extracted collagen complex for anterior cruciate ligament reconstruction.

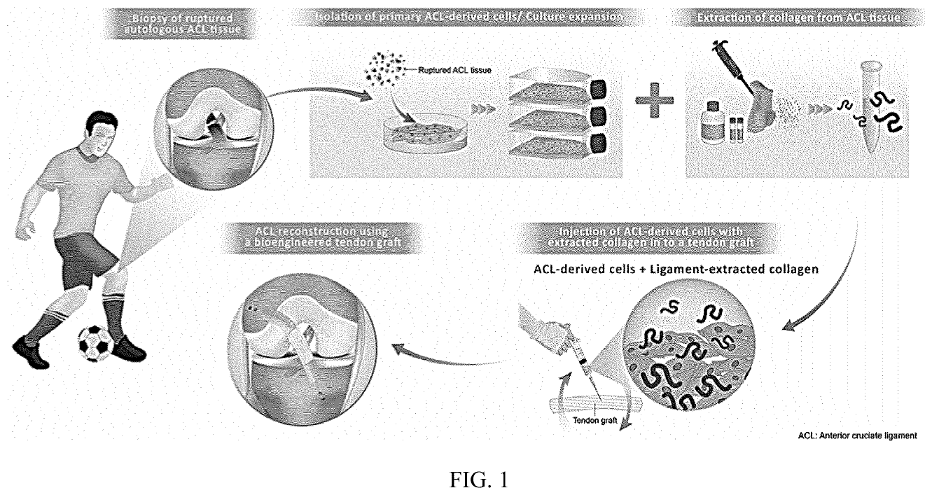

FIG. 2A shows the results of staining anterior cruciate ligament tissue-derived stem cells (ACLSCs) and human adipose-derived mesenchymal stem cells (ADMSCs) with hematoxylin and eosin (H&E) and phalloidin.

FIG. 2B shows the results of confirming the cell adhesion ability of anterior cruciate ligament tissue-derived stem cells (ACLSCs) and human adipose-derived mesenchymal stem cells (ADMSCs).

FIG. 2C shows the results of confirming the cell proliferation rates of anterior cruciate ligament tissue-derived stem cells (ACLSCs) and human adipose-derived mesenchymal stem cells (ADMSCs).

FIG. 2D shows the results of confirming whether mesenchymal stromal cell markers and hematopoiesis-originated stem cells are expressed in anterior cruciate ligament tissue-derived stem cells (ACLSCs) and human adipose-derived mesenchymal stem cells (ADMSCs): mesenchymal stromal cell markers—CD29, CD44, CD90, and CD105; and hematopoiesis-originating stem cells—CD34 and CD45.

FIG. 3A shows the results of confirming cells integrated into tendon matrices after injecting anterior cruciate ligament tissue-derived stem cells (ACLSCs) or human adipose-derived mesenchymal stem cells (ADMSCs) into tendon grafts.

FIG. 3B shows the results of confirming the number of cells integrated into tendon matrices after injecting anterior cruciate ligament tissue-derived stem cells (ACLSCs) or human adipose-derived mesenchymal stem cells (ADMSCs) into tendon grafts.

FIG. 3C shows the results of confirming the number of Ki-67 positive cells after injecting anterior cruciate ligament tissue-derived stem cells (ACLSCs) or human adipose-derived mesenchymal stem cells (ADMSCs) into tendon grafts.

FIG. 3D shows the results of confirming the degree of death of injected cells after injecting anterior cruciate ligament tissue-derived stem cells (ACLSCs) or human adipose-derived mesenchymal stem cells (ADMSCs) into tendon grafts.

FIG. 3E shows the results of confirming the mortality rate of injected cells after injecting anterior cruciate ligament tissue-derived stem cells (ACLSCs) or human adipose-derived mesenchymal stem cells (ADMSCs) into tendon grafts.

FIG. 4A shows the results of confirming type I and III collagens with Sirius Red staining after injecting anterior cruciate ligament tissue-derived stem cells (ACLSCs) or human adipose-derived mesenchymal stem cells (ADMSCs) into tendon grafts.

FIG. 4B shows the results of confirming the levels of type I collagen production with immunofluorescence after injecting anterior cruciate ligament tissue-derived stem cells (ACLSCs) or human adipose-derived mesenchymal stem cells (ADMSCs) into tendon grafts.

FIG. 4C shows the results of confirming the mRNA expression levels of ligament-specific markers COL1, COL3, TNC, and TNMD, a chondrogenic marker COL2, and smooth muscle actin (SMA) after injecting anterior cruciate ligament tissue-derived stem cells (ACLSCs) or human adipose-derived mesenchymal stem cells (ADMSCs) into tendon grafts.

FIGS. 5A and 5B show the results of confirming the level of type I collagen in an extracellular matrix collagen complex extracted from cruciate ligament tissue.

FIG. 6A shows the results of confirming the engraftment ability and viability of cells in the tendon after injecting anterior cruciate ligament tissue-derived stem cells (ACLSCs) alone, type I collagen (COL1) alone, cruciate ligament extracellular matrix-derived collagen (CC) alone, a mixture of anterior cruciate ligament tissue-derived stem cells and type I collagen (COL1+ACLSC), or a mixture of anterior cruciate ligament tissue-derived stem cells and cruciate ligament extracellular matrix-derived collagen (CC+ACLSC) into decellularized tendon grafts.

FIG. 6B shows the results of immunohistochemistry to confirm the engraftment ability and viability (Ki67) of cells in the tendon and the level of type I collagen (COL1) after injecting anterior cruciate ligament tissue-derived stem cells (ACLSCs) alone, type I collagen (COL1) alone, cruciate ligament extracellular matrix-derived collagen (CC) alone, a mixture of anterior cruciate ligament tissue-derived stem cells and type I collagen (COL1+ACLSC), or a mixture of anterior cruciate ligament tissue-derived stem cells and cruciate ligament extracellular matrix-derived collagen (CC+ACLSC) into decellularized tendon grafts.

FIG. 7 shows the results of confirming the engraftment ability and viability of cells in the tendon after mixing anterior cruciate ligament tissue-derived stem cells with an extracellular matrix collagen complex at different concentrations and injecting the resulting mixture into the tendon grafts.

FIG. 8A shows the results of confirming the engraftment ability and viability (Ki67) of cells in the tendon and the level of type I collagen (COL1) after injecting anterior cruciate ligament tissue-derived stem cells alone into fresh tendon grafts.

FIG. 8B shows the results of confirming the engraftment ability and viability (Ki67) of cells in the tendon and the level of type I collagen (COL1) after injecting a mixture of anterior cruciate ligament tissue-derived stem cells and cruciate ligament extracellular matrix-derived collagen into fresh tendon grafts.

FIG. 9A shows the results of confirming the levels of type I collagen (COL1) and the presence or absence of YAP nuclear translocation after injecting anterior cruciate ligament tissue-derived stem cells alone into fresh tendon grafts.

FIG. 9B shows the results of confirming the levels of type I collagen (COL1) and the presence or absence of YAP nuclear translocation after injecting a mixture of anterior cruciate ligament tissue-derived stem cells and cruciate ligament extracellular matrix-derived collagen into fresh tendon grafts.

FIG. 10 schematically shows the effect of YAP nuclear translocation on the ligamentization of tendon grafts.

FIG. 11 shows the results of confirming the engraftment ability and viability (Ki67) of cells in the tendon and the level of type I collagen (COL1) after injecting a mixture of anterior cruciate ligament tissue-derived stem cells and cruciate ligament extracellular matrix-derived collagen into decellularized tendon grafts or fresh tendon grafts.

FIG. 12 shows the results of confirming the engraftment ability and viability (Ki67) of cells in the tendon and the degree of formation of type I collagen (COL1) after injecting a mixture of anterior cruciate ligament tissue-derived stem cells and cruciate ligament extracellular matrix-derived collagen into decellularized tendon grafts or fresh tendon grafts.

FIG. 13 shows the results of comparing the degree of ligamentization after implanting, into an anterior cruciate ligament rupture animal model, tendon grafts alone or tendon grafts in which a mixture of anterior cruciate ligament tissue-derived stem cells and cruciate ligament extracellular matrix-derived collagen was injected.

FIG. 14 shows the results of confirming the engraftment ability and viability (Ki67) of cells in the tendon and the level of type I collagen (COL1) in implantation sites after implanting, into an anterior cruciate ligament rupture animal model, tendon grafts alone or tendon grafts in which a mixture of anterior cruciate ligament tissue-derived stem cells and cruciate ligament extracellular matrix-derived collagen was injected.

FIG. 15 shows the results of implanting, into an anterior cruciate ligament rupture animal model, tendon grafts alone or tendon grafts in which a mixture of anterior cruciate ligament tissue-derived stem cells and cruciate ligament extracellular matrix-derived collagen was injected, then staining implantation sites with Picrosirius red, and confirming type 1 collagen production and the crimp pattern, which is a characteristic of normal ligament tissue, by polarized light microscopy.

FIG. 16 shows the results of comparing the levels of type I collagen (COL1) and YAP nuclear translocation on day 14 after injecting ADMSCs alone, a mixture of ADMSCs+cruciate ligament extracellular matrix collagen (ADMSC+CC), ACLSCs alone, or a mixture of ACLSCs+cruciate ligament extracellular matrix collagen (ACLSC+CC) into decellularized tendon grafts.

FIG. 17 shows the results of comparing the levels of type I collagen (COL1) and YAP nuclear translocation on day 14 after injecting a mixture of ADMSCs+cruciate ligament extracellular matrix collagen (ADMSC+CC), ACLSCs alone, or a mixture of ACLSCs+cruciate ligament extracellular matrix collagen complex (ACLSC+CC) into decellularized tendon grafts.

### MODES OF THE INVENTION

Hereinafter, one or more specific exemplary embodiments will be described in more detail through examples. However, these examples are provided only for exemplarily explaining the one or more specific exemplary embodiments, and the scope of the present invention is not limited to these examples.

### Experimental Method

### Collection of Anterior Cruciate Ligament Tissue and Isolation of Stem Cells

Portions of damaged anterior cruciate ligament tissue were collected from 14 patients (11 men, 3 women) with an average age of 22.7±4.25 years who underwent primary anterior cruciate ligament reconstruction surgeries within 4 weeks of anterior cruciate ligament (ACL) rupture. An average of 937 mg (range from 598 mg to 1,431 mg) of anterior cruciate ligament tissue was collected.

The collected anterior cruciate ligament tissue was cut into fragments of 1 cm or less using scissors. The fragments were placed in serum-free DMEM containing a penicillin-streptomycin antibiotic (Thermo Fisher, 15140122) and 1 mg/ml collagenase type I (Gibco, 17100-017: Sigma, C0130) to dissolve an extracellular matrix at 37° C. for 16 to 20 hours. DMEM was filtered through a 70 μm cell strainer to recover cells that had passed through the cell strainer, and the cells were washed several times with serum-free DMEM. Thereafter, the cells were re-suspended in 4 ml of PBS in a sterile tube for subsequent experiments. After centrifugation at 1,500 rpm for 3 min, the cells were stored at −80° C. in a deep freezer. Hereinafter, stem cells derived from anterior cruciate ligament tissue will be described as anterior cruciate ligament-derived stem cells (ACLSCs).

Human adipose-derived mesenchymal stem cells (ADMSCs) were purchased from a cell bank (CEFO Co., Seoul, Korea) and cultured and stored in the same manner as the ACLSCs. The ADMSCs used in the experiment were isolated from adipose tissue taken from a 25-year-old female donor (Cefobio, CB-ADMSC-001).

All cells were checked for the presence of mycoplasma by a PCR-based method (Takara, 6601) before long-term storage in liquid nitrogen. For both ACLSCs and ADMSCs, cells with a passage number of 3 were used in the experiment, and cells from a single donor were used for each experimental set.

### 2. Cell Adhesion Assay

To compare the adhesion of cells, a total of 1×10⁵ cells were seeded in sextuplicate in each well of a 6-well plate and cultured for one day. Cells not adhered to the plate were removed by carefully washing three times with DMEM, and the cells were fixed with 10% formalin for 10 minutes. Thereafter, the cells were stained with a Giemsa staining solution (Sigma, 48900) for 15 minutes. Adhered cells were imaged and counted using an upright microscope. To stain F-actin with phalloidin, cells were fixed with 10% formalin for 10 minutes and permeabilized with 3% Triton X-100 for 15 minutes. Afterward, the cells were washed twice with 1×PBS and stained with an F-actin solution for 20 min. The cells were washed twice with 1×PBS. The stained cells were imaged using a fluorescence microscope.

### 3. Cell Proliferation Assay

Cell proliferation levels were evaluated using water-soluble tetrazolium salt (WST: DoGen, EZ-1000) according to the manufacturer's instructions. Cells were seeded at a density of 1×10³ cells/well in 96-well plates in triplicate. On the day of the experiment, 10 μl of WST solution was added to each well and incubated for 1 hour to allow WST to be metabolized to formazan. Absorbance was measured at 450 nm using a microplate plate reader.

### 4. Fluorescence-Activated Cell Sorting (FACS)

Cells were fixed with 2% formalin and stained with CD29-PE (Biolegend, 303004), CD34-APC (Biolegend, 343607), CD44-PE (Biolegend, 338807), CD45-APC (Biolegend, 368511), CD90-PE (Biolegend, 328109) or CD105-PE (Biolegend, 400112) at 4° C. for 10 minutes. After staining, the cells were washed with 1×PBS containing 0.5% BSA and 0.1% sodium azide. Flow cytometry was performed using a FACS Canto II (B.D. Biosciences, San Jose, CA, USA), and data was analyzed with Flow Jo software (Tracstar, Ashland, OR, USA).

5. Extraction of Extracellular Matrix Complex from Human Allogeneic Cruciate Ligament Tissue

### 5-1. Removal of Non-Collagenous Solubilized Material

A non-collagenous solubilized material was removed from collected anterior and posterior cruciate ligament tissues. Specifically, about 1 g of cruciate ligament tissue and 10 ml of cold 0.5 M sodium acetate were placed in a 50 ml tube, and the cruciate ligament tissue mixture was homogenized for 1 minute using a bench top tissue homogenizer (2. Bench top homogenizer) set at 6 m/sec. After centrifugation, the supernatant in the tube was discarded and the homogenization process was repeated once more. To remove any remaining sodium acetate in the ligament tissue, the residue was washed with cold tertiary distilled water. After removing as much liquid as possible from the tube, the cruciate ligament tissue was transferred to a new tube.

### 5-2. Collagen Extraction

A 0.075 M sodium citrate buffer was added to the tube containing the cruciate ligament tissue at a volume of 2 ml/g, and the tissue was homogenized for 1 minute using a tissue homogenizer set at 6 m/sec. After centrifugation, the supernatant in the tube was discarded and the homogenization process was repeated once more. The homogenate was centrifuged, the supernatant in the tube was discarded, and 4 ml of fresh 0.075 M sodium citrate buffer was added. The resulting mixture was homogenized for 6 minutes using a tissue homogenizer set at 6 m/sec, and then centrifuged. The supernatant in the tube was transferred to a new tube, and 1 ml of fresh 0.075 M sodium citrate buffer was added. The resulting mixture was homogenized for 5 minutes using a tissue homogenizer set at 6 m/sec, and then centrifuged. The supernatant was transferred to a new 15 ml tube and centrifuged at 3,200×g at 4° C. for 30 minutes. The final supernatant was filtered through a 0.45 μm pore size filter and then stored at 4° C. in a refrigerator.

6. Injection of ACLSCs or ACLSCs+Collagen into Decellularized Tendon Grafts

### 6-1. Preparation of Decellularized Tendon Graft and Injection of ACLSCs

Fourteen human decellularized tibialis tendon allografts (hereafter referred to as tendon grafts) were donated. The donated tendon grafts were processed as follows. The tendon grafts were completely removed from the muscle and washed with sterile distilled water for 5 minutes. Thereafter, the tendon grafts were treated with 3% hydrogen peroxide for 5 minutes and 70% ethyl alcohol for 15 minutes. All washing procedures were repeated three times. For sterilization, the tendon grafts were irradiated with low doses (25 kGy) of gamma rays and then frozen and stored at −80° C. For the experiment, the tendon grafts were thawed at room temperature for 15 minutes, and then only a transitional zone was selected and cut into pieces with a length of about 1.5 cm. Tendon grafts (for example: tibialis tendons of both legs) from a single donor were used for each experimental set.

ACLSCs or ADMSCs with a passage number of 3 were treated with trypsin and collected as suspended cells. The cells were stained with trypan blue to count the viable cells, and 200 μl of a cell suspension containing 2×10⁶ cells was prepared. The cells were seeded into the fragments of the tendon grafts by injecting the cell suspension directly into the tendon grafts using a 25 gauge needle. The cells were injected into the center part of the graft, and cell leakage was carefully monitored. The graft fragments injected with the cell suspension were placed in a 5% CO₂ incubator for an initial 20 minutes, and then supplemented with a DMEM (high glucose, Hyclone, SH30243.01) medium containing 10% FBS and 1% penicillin-streptomycin. The DMEM growth medium was replaced every 3 days.

On designated dates (on days 1, 3 and 7 after cell injection), the graft fragments were collected, completely embedded in the Tissue-Tek O.C.T compound (SAKURA, 4583), and stored at −80° C. Thereafter, frozen tendon graft fragments were cut longitudinally into 6 μm thicknesses to prepare frozen tissue sections. Tissue sections were used for histological staining and immunochemical examination, adhered to glass slides and stored at −80° C.

### 6-2. ACLSC+Collagen Injection

The same tendon graft as in 6-1 above was used, but ACLSCs (2×10⁶ cells) and collagen (9 mg/ml) were mixed in the tendon grafts and injected using a single syringe. The concentration of collagen was adjusted with DMEM containing 10% FBS and 1% penicillin-streptomycin, and the mixture was cultured and frozen tissue sections were prepared in the same manner as in 6-1 above.

7. Injection of ACLSCs or ACLSCs+Collagen into Fresh Grafts

Fresh human gracilis tendons generated during anterior cruciate ligament surgery were collected to prepare grafts with the same size as in 6-1, and cell and collagen injection experiments were performed in the same manner as in 6-1 and 6-2 within 4 hours of tissue collection.

8. Implantation of Mixture of ACLSCs and Extracellular Matrix Collagen into Anterior Cruciate Ligament Animal Model

The experimental groups were set as a control, a cruciate ligament extracellular matrix collagen complex, or ACLSCs+cruciate ligament extracellular matrix collagen complex (9 mg/ml). Twenty rats were bred in each group (10 rats each were evaluated on weeks 4 and 8 after surgery). ACLSCs with a passage number of 3 were prepared per experimental group at a concentration of 3×10⁵ cells/10 μl. A mixture of 30 μl of DMEM (containing ACLSCs)+collagen (9 mg/ml) was prepared for use in the ACLSC+collagen complex injection experimental group.

The tendon grafts were collected from the transitional zones of the Achilles tendons of both ankles of 8-week-old rats and stored in an ultra-low temperature freezer in advance. One day before anterior cruciate ligament reconstruction surgery in rats, the tendon grafts stored in the ultra-low temperature freezer were thawed, and the cruciate ligament extracellular matrix collagen complex or ACLSC+cruciate ligament extracellular matrix collagen complex was injected into the Achilles tendon distal portion to be used for surgery using a 31G insulin syringe, and then the tendon grafts were left to stand in a CO₂ incubator for 24 hours. Thereafter, anterior cruciate ligament reconstruction surgery was performed on the left hind limbs of the rats, and at the designated time (4 or 8 weeks after surgery), the rats were sacrificed to collect hind limb tissue. Among the tissues obtained from each group, five were immediately stored in an ultra-low temperature freezer after clearing of surrounding tissue for use in mechanical analysis, and five were fixed in 10% formalin for use in histological analysis.

### 9. Histological Analysis

Histological staining was performed according to standard protocols or the manufacturer's instructions. Slides were stained with hematoxylin and eosin (H&E) for histological analysis, Masson's trichrome (Abeam, ab150686) for total collagen analysis, and Picrosirius red (Abeam, ab150681) for type I and III collagen analysis. All slides were mounted with a permanent mounting medium (Vector Lab, H-5000). The cell-injected regions in each slide were observed at 200× magnification in high power fields. The number of stained positive cells was counted in three sites of five consecutive slides. Measurements were performed by two blinded independent observers, and mean values were used for analysis.

### 10. Immunohistochemistry (IHC) Analysis

Tissue slides were fixed with cold acetone and then washed three times with distilled water and 1×PBS. Thereafter, tissue slides were incubated in 1×PBS containing 0.3% Triton X-100 for 10 minutes and in BLOXALL (Vector Lab, SP-6000) for 30 minutes.

Tissue slides were immunostained with primary antibodies diluted 1:100 in an antibody diluent (DAKO, S3022) at room temperature for 1 hour, and then incubated with a biotinylated anti-rabbit IgG secondary antibody (Vector Lab, BA-1000) mixed with normal horse serum (Vector Lab, S-2012) for 1 hour. Thereafter, the tissue slides were incubated with an ABC kit component (Vector Lab, PK-6102) and incubated with a DAB substrate kit (Vector Lab, sk4100) for 1 to 5 minutes. Immunostained slides were washed five times with distilled water and then counterstained with hematoxylin (Merck, 1.05174.0500) for 10 seconds. Slides were allowed to pass through graded ethanol solutions (50% to 100% ethanol) to dehydrate the tissue and finally mounted with a permanent mounting medium (Vector Lab, H-5000). Images of stained cells were confirmed under a Nikon eclipse Ti-U microscope. Antibodies used for IHC were as follows: COL1A1 (collagen, type I, alpha 1) (Santa, 8784; diluted 1:200), and COL3 (collagen type III) (Abclonal, a3795; diluted 1:200).

### 11. Immunofluorescence (IF)

Tissue slides were fixed with 4% paraformaldehyde for 15 minutes and permeabilized with 1×PBS containing 0.3% Triton X-100 and 1% BSA (Rocky Mountain Biologicals, Inc, MT, USA) for 1 hour. Thereafter, the slides were stained with primary antibodies at 4° C. overnight. The next day, the slides were washed twice with 1×PBS and incubated with Cy3- or Alexa 488-conjugated secondary antibodies at room temperature for 1 hour. The slides were washed with distilled water and mounted with DAPI (Vector CA, USA). Tissue images were obtained under a confocal microscope. Antibodies used for IF were Ki-67 (Invitrogen, 14-5698-82) or collagen type 1 (Santa, sc-8784 and Cell Signaling Technology, 66948) antibodies.

### 12. RT-qPCR

For mRNA analysis, tissue was collected from frozen tissue slides with a scraper and total RNA was extracted with TRIZOL reagent. A total of 1 μg of cDNA was synthesized using reverse transcriptase (Thermo Scientific, EP0442) and used as a template for RT-qPCR. RT-qPCR primers are shown in Table 1: COL1=collagen type I; COL2=collagen type II; COL3=collagen type III; TNC=tenascin C; TNMD=tenomodulin; SMA=smooth muscle actin; and GAPDH=glyceraldehyde 3-phosphate de hydrogenase.

TABLE 1

| Classification | | Primer sequence |
|---|---|---|
| COL1 | forward | 5′-AGTGGTTTGGATGGTGCCAA-3′ |
| | reverse | 5′-GCACCATCATTTCCACGAGC-3′ |
| COL2 | forward | 5′-CAACCAGGACCAAAGGGACA-3′ |
| | reverse | 5′-ACCTTTGTCACCACGATCCC-3′ |
| COL3 | forward | 5′-CTTCTCTCCAGCCGAGCTTC-3′ |
| | reverse | 5′-CCAGTGTGTTTCGTGCAACC-3′ |
| TNC | forward | 5′-GGTTGCTGGAGACTGTGGAA-3′ |
| | reverse | 5′-AGGTTTTCCAGAAGGGGCAG-3′ |
| TNMD | forward | 5′-AATGAACAGTGGGTGGTCCC-3′ |
| | reverse | 5′-TTGCCTCGACGGCAGTAAAT-3′ |
| SMA | forward | 5′-GTGATGGTGGGAATGGGACAA-3′ |
| | reverse | 5′-AGTGGTGCCAGATCTTTTCCA-3′ |
| GAPDH | forward | 5′-CAAGATCATCAGCAATGCC-3′ |
| | reverse | 5′-CTGTGGTCATGAGTCCTTCC-3′ |

### 13. Statistical Analysis

Graph-Pad Prism 7.0 was used for quantitative data and statistical analysis. Statistical significance was tested appropriately using the Mann-Whitney U test and one-way analysis of variance (ANOVA). Experimental result values are expressed as the mean±standard error (SEM) obtained from at least three independent experiments. Asterisks indicate the statistical significance levels (*p<0.05, **p<0.01, ***p<0.001).

### Experimental Results

### Cellular Characterization of ACLSCs and ADMSCs

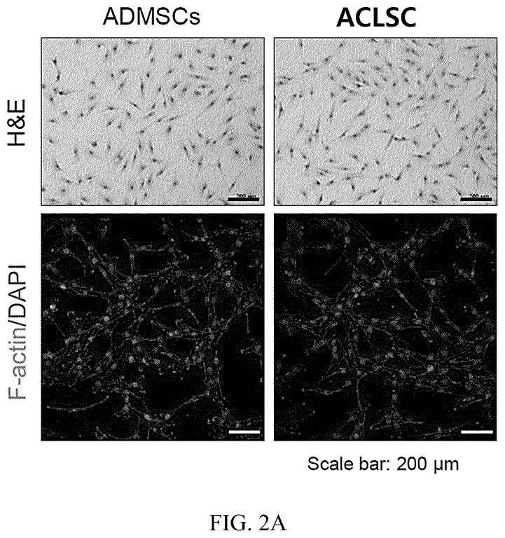

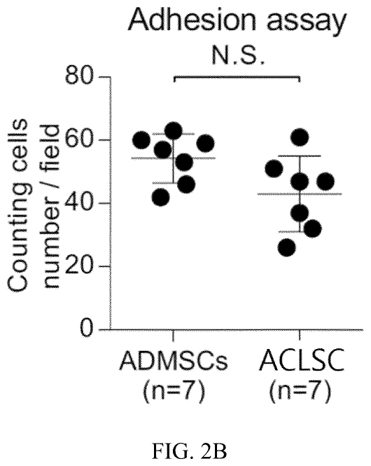

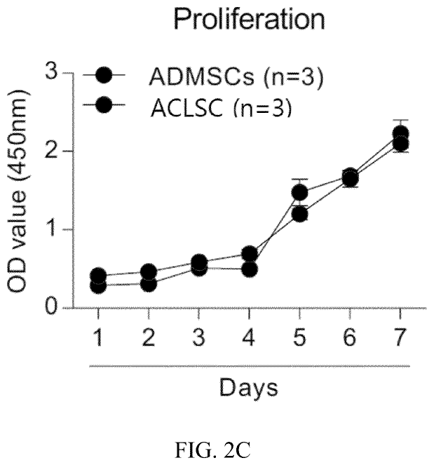

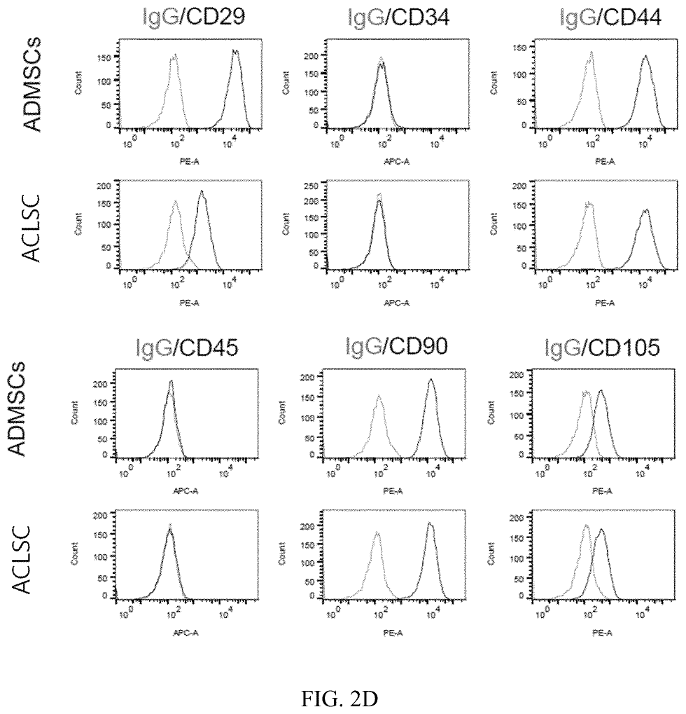

Both ACLSCs and ADMSCs showed a typical elongated spindle-like morphology in H&E and F-actin staining (FIG. 2A). As a result of cell adhesion and proliferation experiments, ACLSCs and ADMSCs showed similar adhesion and proliferation rates (FIGS. 2B and 2C).

As a result of FACS analysis, both cell groups expressed mesenchymal stromal cell markers CD29, CD44, CD90, and CD105, but did not express hematopoietic stem cell markers CD34 and CD45 (FIG. 2D).

2. Confirmation of Migration and Viability of ACLSCs Injected into Tendon Grafts

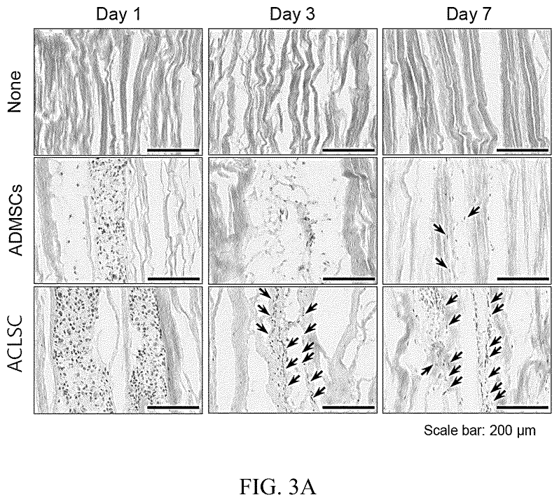

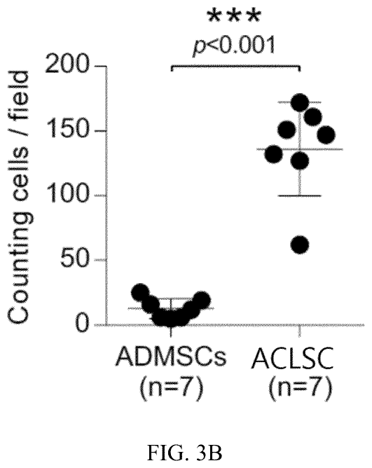

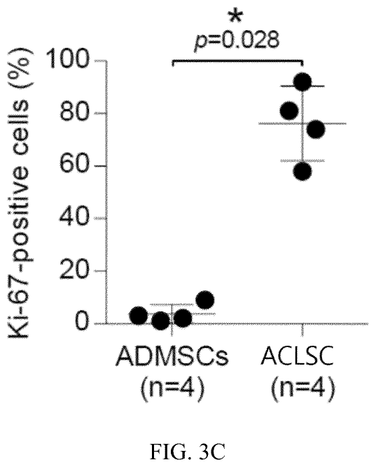

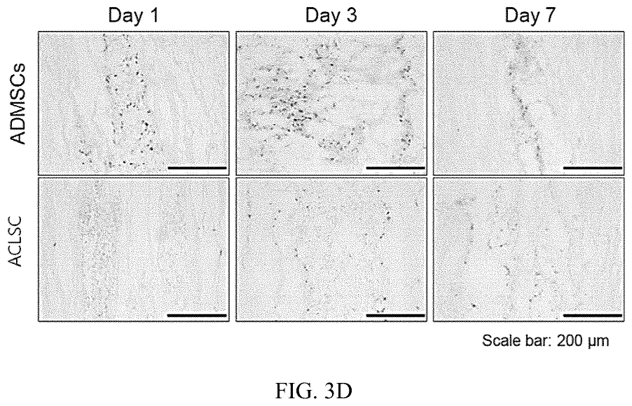

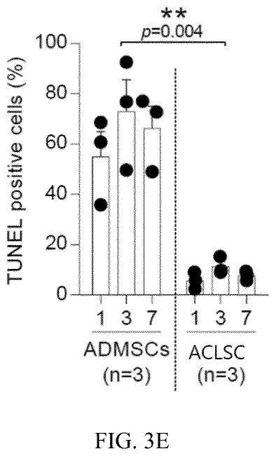

As a result of H&E staining, cell clusters could be seen along the intrafascicular openings within the cell-injected fibers on day 1 after the injection of cells into tendon grafts (FIG. 3A). Further, in the ACLSC-injected tendon grafts, numerous elongated cells integrated into the matrix could be clearly confirmed on days 3 and 7. However, in the ADMSC-injected tendon grafts, it could be observed that only a small number of cells were integrated into the matrix (FIG. 3A). The number of cells integrated into the tendon matrix was counted on day 7, and it was confirmed that the number of integrated cells was significantly higher in the ACLSC-injected tendon grafts than in the ADMSC-injected tendon grafts (FIG. 3B) (P=0.006).

In addition, the proliferation ability of the cells injected into the tendon grafts was confirmed by Ki-67 immunohistochemical staining, and it was confirmed that the number of Ki-67 positive cells was significantly higher in the ACLSC-injected tendon grafts than in the ADMSC-injected tendon grafts on day 7 of the experiment (FIG. 3C) (P=0.028).

Conversely, cell death rates evaluated by TUNEL analysis were significantly higher in the ADMSC-injected tendon grafts than in the ACLSC-injected tendon grafts (FIGS. 3D and 3E) (P=0.004).

### 3. Confirmation of Protein Content and Gene Expression Levels of ACLSC-Injected Tendon Grafts

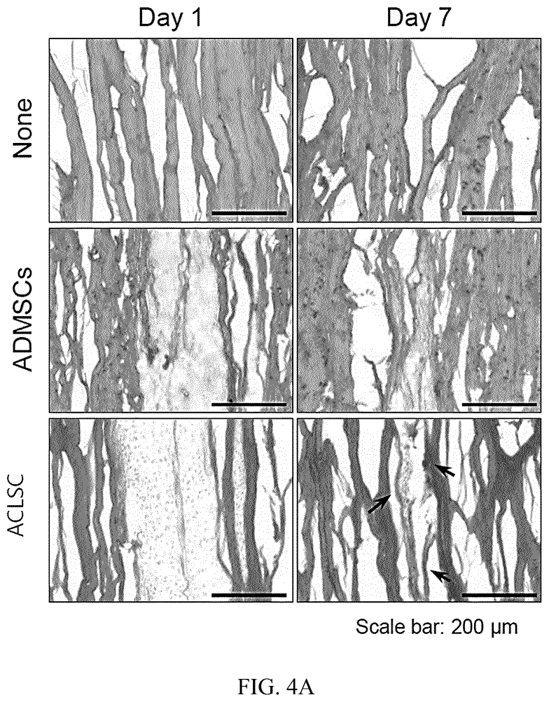

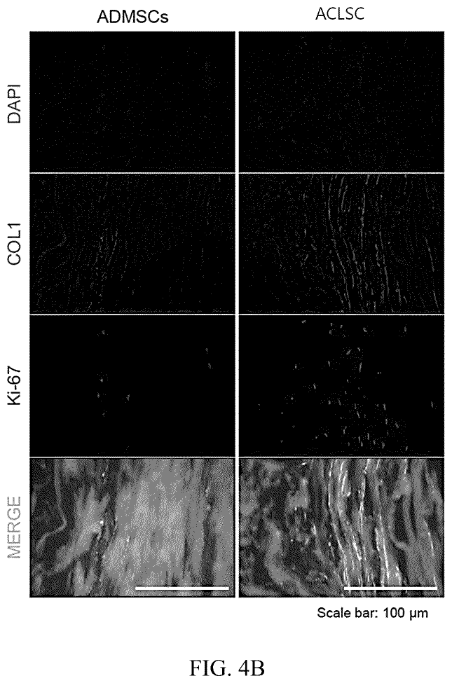

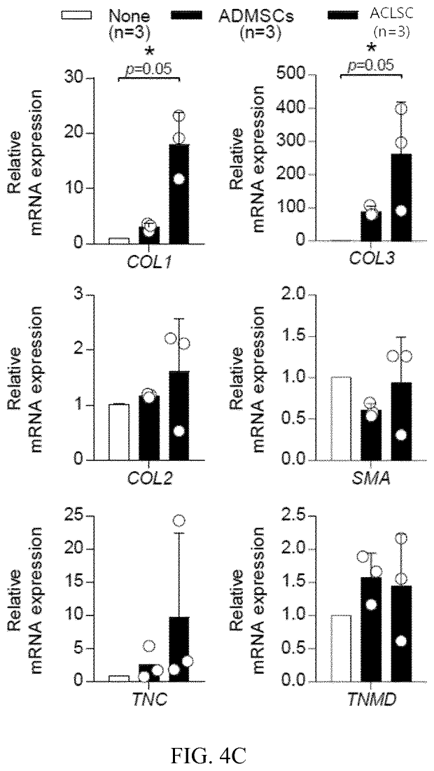

As a result of comparing the degree of collagen deposition in the tendon grafts using Masson's trichrome staining and Sirius Red staining, it was confirmed that collagen deposition was more consistent in the ACLSC-injected tendon grafts than in the ADMSC-injected tendon grafts on day 7 of the experiment (FIG. 4A).

Furthermore, it was confirmed through immunofluorescence that Ki-67-positive ACLSCs integrated into the matrix of the tendon grafts remarkably produced type I collagen (FIG. 4B).

mRNA expression analysis performed on day 7 after cell injection showed that gene expression levels of types I and III collagen were significantly higher in the ACLSC-injected tendon grafts than in the ADMSC-injected tendon grafts. However, the expression of other ligament-specific markers including tenascin C (TNC), tenomodulin (TNMD) and smooth muscle actin (SMA) did not show significant differences (FIG. 4C).

4. Confirmation of Decellularized Tendon Grafts Implanted with Mixture of ACLSCs and Cruciate Ligament Extracellular Matrix Collagen

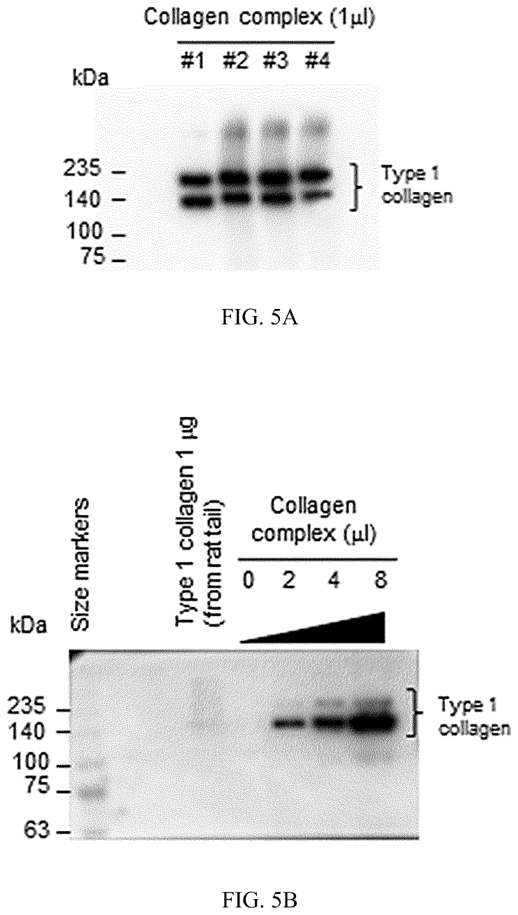

As a result of confirming the level of type I collagen in an extracellular matrix collagen complex extracted from cruciate ligament tissue, it was confirmed that type I collagen was the main component (FIG. 5A). In addition, even when the extracellular matrix collagen complex was extracted from the cruciate ligament tissue at different concentrations and then the level of type I collagen was confirmed, it was confirmed that type I collagen was included (FIG. 5B).

ACLSCs alone (ACLSC), type I collagen alone (COL1), anterior cruciate ligament extracellular matrix collagen complex alone (CC), a mixture of ACLSCs+type I collagen (COL1+ACLSC), or a mixture of ACLSCs+extracellular matrix collagen complex (CC+ACLSC) was implanted into decellularized tendon grafts. On day 7 after implantation, the engraftment ability and viability of cells in the tendon were confirmed by H&E staining, and the level of type I collagen was confirmed by immunofluorescence staining.

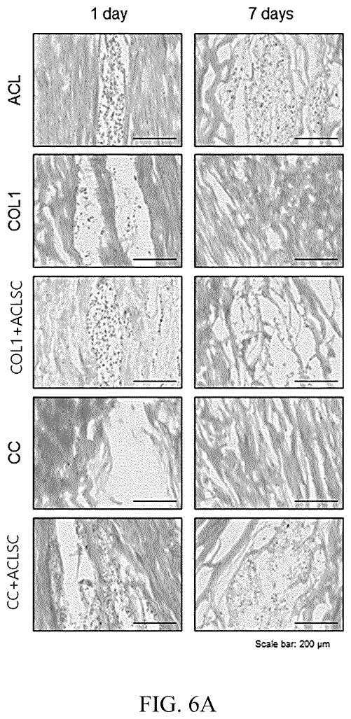

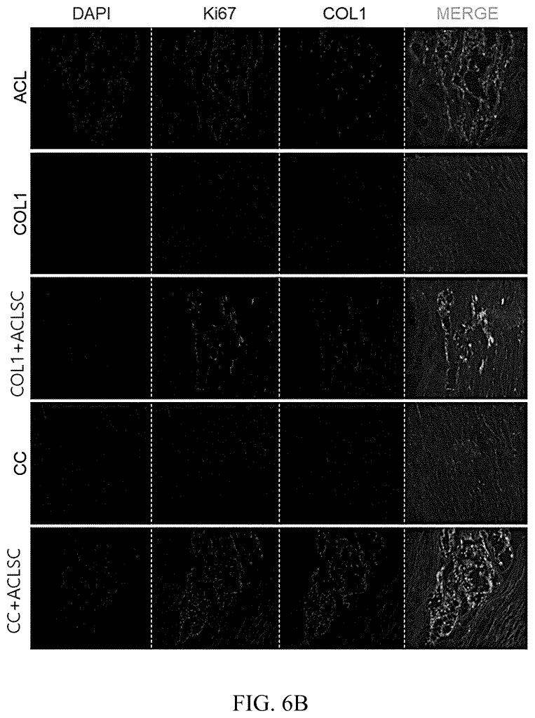

As a result, it could be seen that the injection of a mixture of ACLSCs+extracellular matrix collagen complex (CC+ACLSC) showed the highest engraftment ability and viability of cells in the tendon compared to other experimental groups (FIG. 6A). Through immunofluorescence staining, it could be confirmed that the formation of type I collagen was highest when the mixture of ACLSCs+extracellular matrix collagen complex (CC+ACLSC) was injected compared to other experimental groups (FIG. 6B).

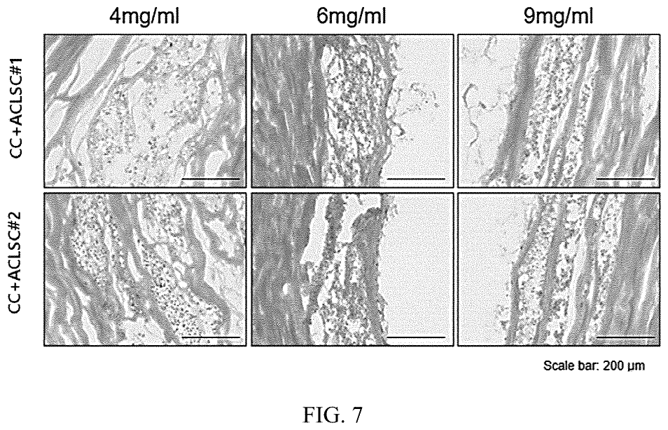

As a result of mixing ACLSCs with an extracellular matrix collagen complex at different concentrations and injecting the resulting mixture into tendon grafts, it was confirmed that ACLSCs had excellent cell engraftment ability and viability when the concentration of the extracellular matrix collagen complex was 6 mg/ml or more, and the best results were shown at a concentration of 9 mg/ml (FIG. 7).

5. Confirmation of Fresh Tendon Grafts Implanted with Mixture of ACLSCs and Cruciate Ligament Extracellular Matrix Collagen

After ACLSCs alone or a mixture of ACLSCs+cruciate ligament extracellular matrix collagen complex was injected into fresh, non-frozen or non-sterile tendon grafts, the engraftment ability and viability of cells in the tendon and the level of type I collagen were confirmed on day 14.

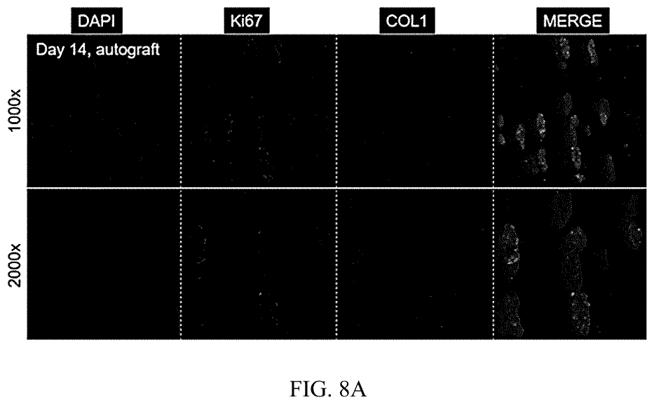

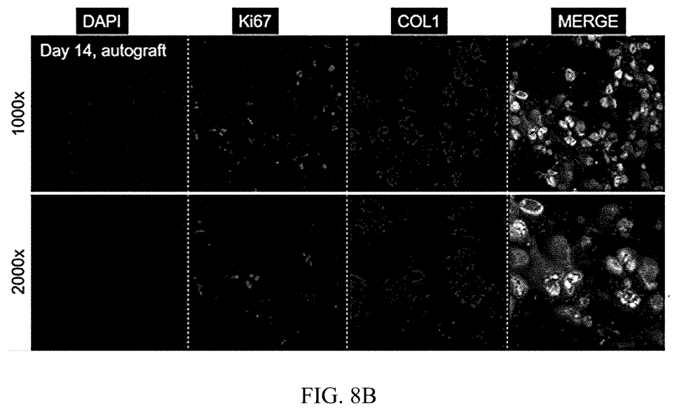

As a result, it could be seen that a group injected with ACLSCs alone and an experimental group injected with a mixture of ACLSCs+cruciate ligament extracellular matrix collagen complex showed excellent engraftment ability and viability of cells in the tendon, and also showed high levels of type I collagen (FIG. 8).

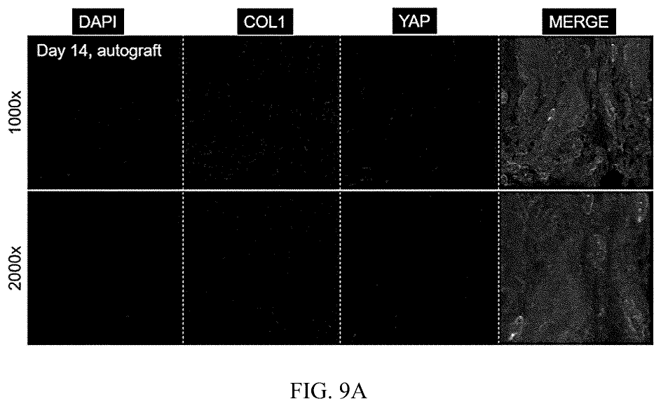

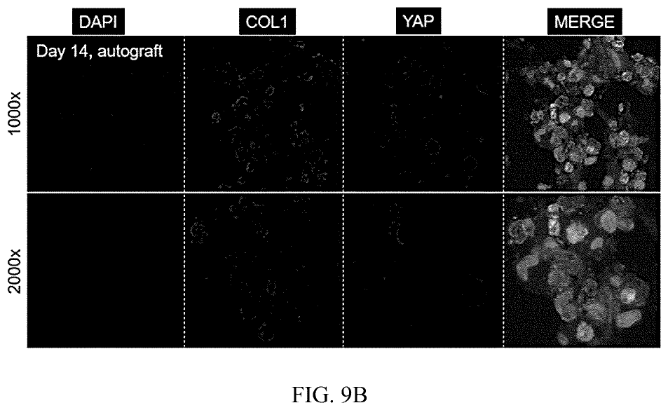

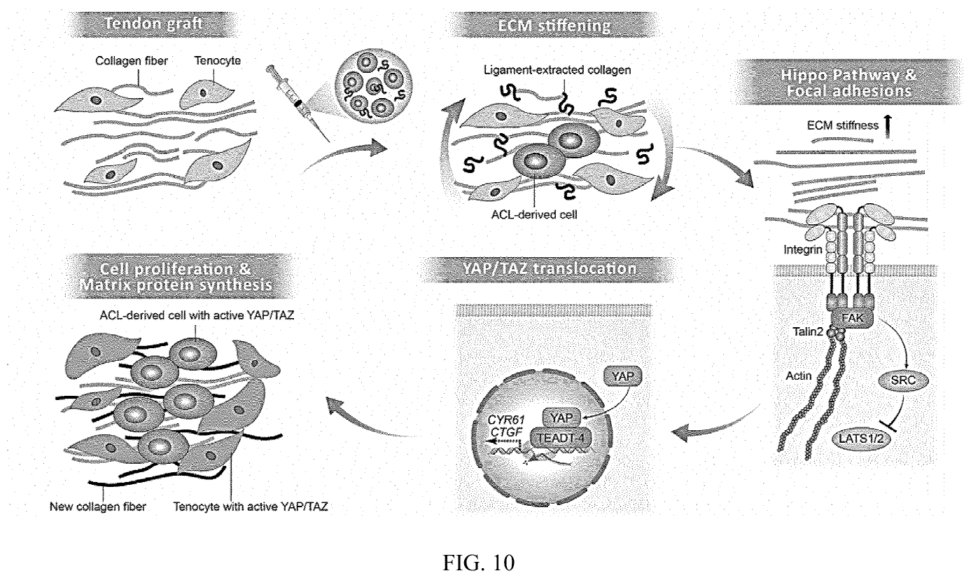

In addition, it was confirmed that the injection of a mixture of ACLSCs+extracellular matrix collagen complex into fresh tendon grafts increased the nuclear translocation of yes-associated protein (YAP) (FIG. 9). This indicates that dynamic interactions occurred between the tendon cells in the freshly implanted tendon and the injected ACLSC+extracellular matrix collagen complex (FIG. 10).

6. Comparison of Decellularized Tendon Grafts with Fresh Tendon Grafts

After a mixture of ACLSCs+cruciate ligament extracellular matrix collagen complex was injected into fresh, non-frozen or non-sterile tendon grafts or decellularized tendon grafts, the engraftment ability and viability of cells in the tendon (Ki67) and the level of type I collagen (COL1) were confirmed on day 14.

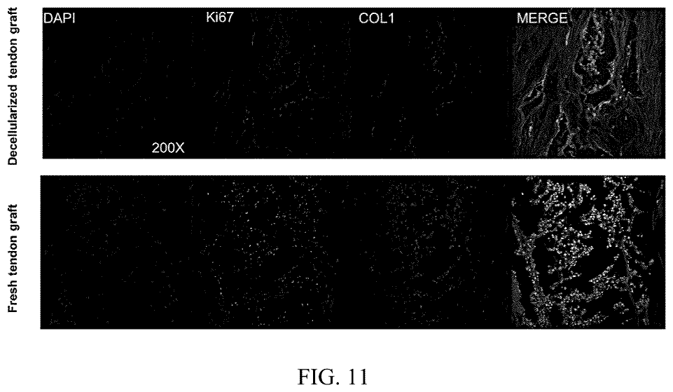

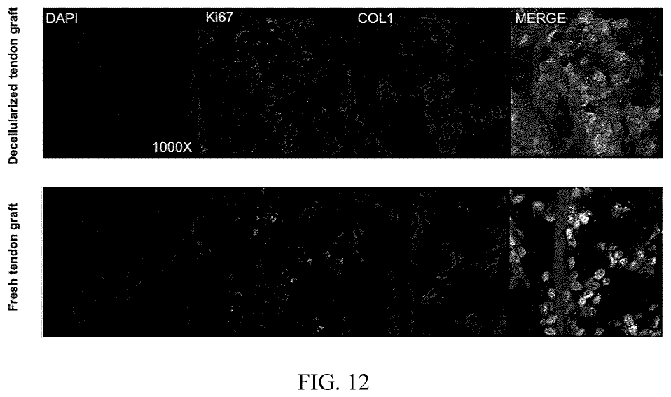

As a result of confirmation, it could be observed that a group implanted with fresh tendon grafts showed better engraftment ability and viability of cells in the tendon, and type I collagen production was also excellent (FIGS. 11 and 12).

### 7. Tendon Graft Implantation in Anterior Cruciate Ligament Rupture Animal Model

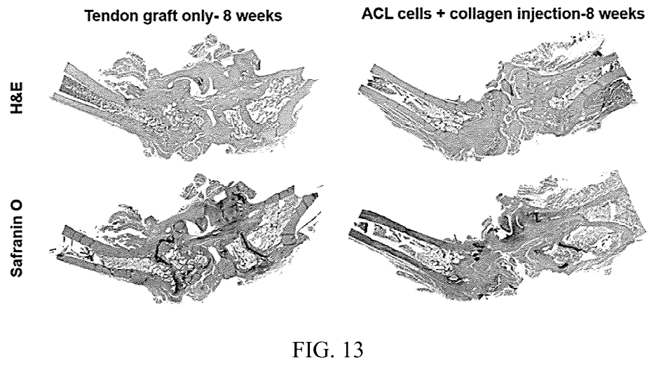

As a result of confirming the effects of tendon grafts alone or tendon grafts injected with a mixture of ACLSCs+extracellular matrix collagen complex in rats with anterior cruciate ligament rupture, it could be confirmed that compared to an experimental group into which tendon grafts were implanted alone (control), ligamentization was excellent in the experimental group in which the mixture of ACLSCs+extracellular matrix collagen complex was injected (FIG. 13).

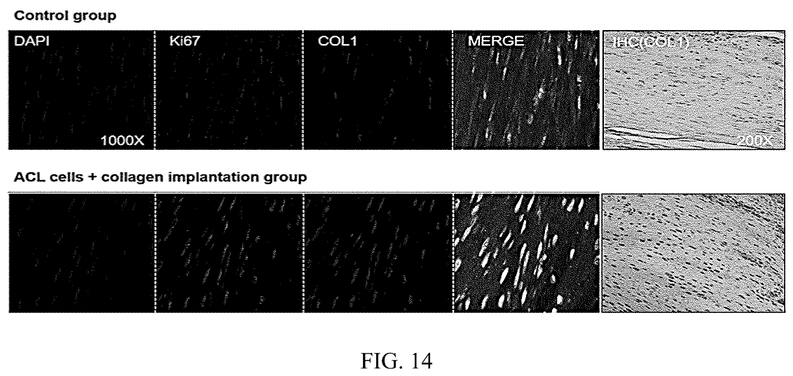

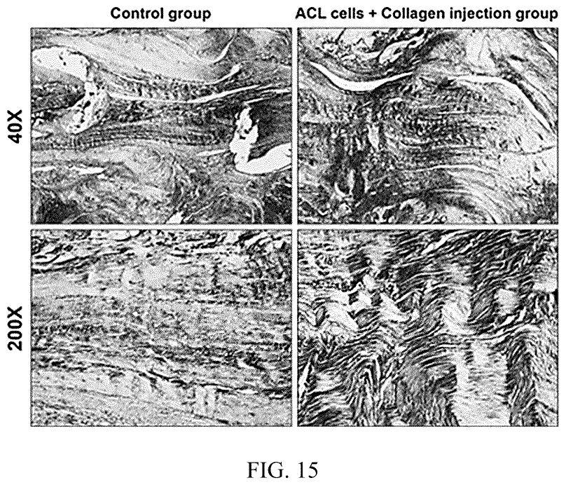

Furthermore, it could be seen that the experimental group in which the mixture of ACLSCs+extracellular matrix collagen complex was injected showed excellent engraftment ability and viability (Ki67) and type I collagen (COL1) levels of cells in the tendon at the implantation site compared to the control (FIG. 14). Even in the results of imaging by polarized light microscopy stained with Picrosirius red, excellent collagen formation and restoration of the crimp pattern could be confirmed in the experimental group into which the mixture of ACLSCs+extracellular matrix collagen complex was injected (FIG. 15).

### 8. Comparison of Effects According to Types of Stem Cells

After ADMSCs alone, a mixture of ADMSCs+cruciate ligament extracellular matrix collagen complex (ADMSC+CC), ACLSCs alone, or a mixture of ACLSCs+cruciate ligament extracellular matrix collagen complex (ACLSC+CC) was injected into decellularized tendon grafts, type I collagen (COL1) levels and YAP nuclear translocation were compared on day 14.

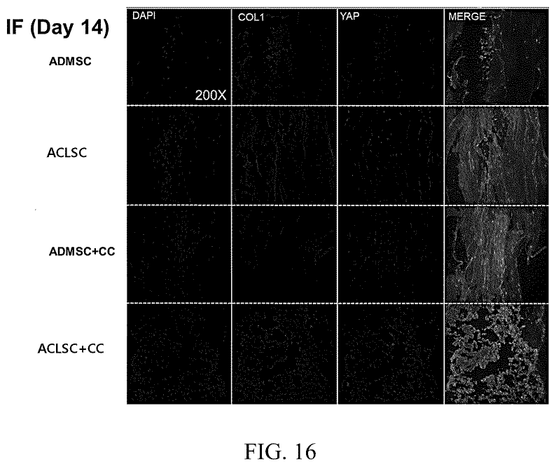

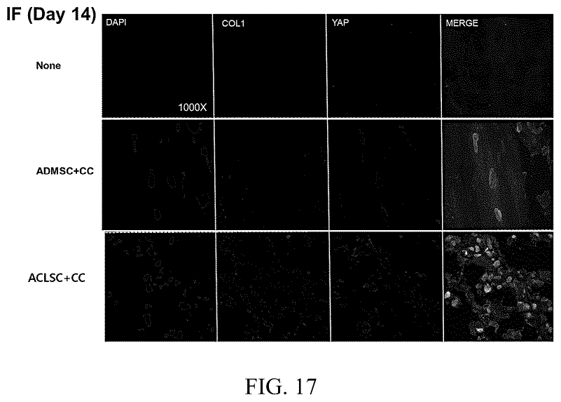

As a result of the comparison, it could be seen that the group in which the mixture of ACLSC+extracellular matrix collagen complex (ACLSC+CC) was injected had the highest type I collagen (COL1) level and YAP nuclear translocation (FIGS. 16 and 17).

1. A method for treating anterior cruciate ligament rupture or regenerating anterior cruciate ligament, comprising the step of transplanting a pharmaceutical composition for treating anterior cruciate ligament rupture into a subject in need thereof, wherein the pharmaceutical composition comprises:
comprising a tendon for a graft; and
anterior cruciate ligament-derived stem cells injected into the tendon.

2. The method of claim 1, wherein the anterior cruciate ligament is autologous or allogeneic.

3. The method of claim 1, wherein the anterior cruciate ligament-derived stem cells are CD34-negative.

4. The method of claim 1, wherein the composition further comprises an allogeneic cruciate ligament-derived collagen complex.

5. The method of claim 4, wherein the collagen complex comprises type I collagen.

6. A method for treating anterior cruciate ligament rupture or regenerating anterior cruciate ligament, comprising administering to a subject in need thereof a composition for inducing ligament regeneration of a graft, said pharmaceutical composition comprising:
Anterior cruciate ligament-derived stem cells; and
Cruciate ligament-derived collagen complex.

7. The method of claim 6, wherein the anterior cruciate ligament is autologous or allogeneic.

8. The method of claim 6, wherein the collagen complex comprises type I collagen.

9. The method of claim 1, wherein the cruciate ligament is allogenic.

10-14. (canceled)


SEQUENCE LISTING (14 sequences)

SEQ ID NO: 1 - DNA, 20 bp, synthetic construct
  source 1..20: mol_type = other DNA
agtggtttggatggtgccaa

SEQ ID NO: 2 - DNA, 20 bp, synthetic construct
  source 1..20: mol_type = other DNA
gcaccatcatttccacgagc

SEQ ID NO: 3 - DNA, 20 bp, synthetic construct
  source 1..20: mol_type = other DNA
caaccaggaccaaagggaca

SEQ ID NO: 4 - DNA, 20 bp, synthetic construct
  source 1..20: mol_type = other DNA
acctttgtcaccacgatccc

SEQ ID NO: 5 - DNA, 20 bp, synthetic construct
  source 1..20: mol_type = other DNA
cttctctccagccgagcttc

SEQ ID NO: 6 - DNA, 20 bp, synthetic construct
  source 1..20: mol_type = other DNA
ccagtgtgtttcgtgcaacc

SEQ ID NO: 7 - DNA, 20 bp, synthetic construct
  source 1..20: mol_type = other DNA
ggttgctggagactgtggaa

SEQ ID NO: 8 - DNA, 20 bp, synthetic construct
  source 1..20: mol_type = other DNA
aggttttccagaaggggcag

SEQ ID NO: 9 - DNA, 20 bp, synthetic construct
  source 1..20: mol_type = other DNA
aatgaacagtgggtggtccc

SEQ ID NO: 10 - DNA, 20 bp, synthetic construct
  source 1..20: mol_type = other DNA
ttgcctcgacggcagtaaat

SEQ ID NO: 11 - DNA, 21 bp, synthetic construct
  source 1..21: mol_type = other DNA
gtgatggtgggaatgggacaa

SEQ ID NO: 12 - DNA, 21 bp, synthetic construct
  source 1..21: mol_type = other DNA
agtggtgccagatcttttcca

SEQ ID NO: 13 - DNA, 19 bp, synthetic construct
  source 1..19: mol_type = other DNA
caagatcatcagcaatgcc

SEQ ID NO: 14 - DNA, 20 bp, synthetic construct
  source 1..20: mol_type = other DNA
ctgtggtcatgagtccttcc